(19) United States
(12) Patent Application Publication
(10) Pub. No.: US 2026/0235591 A1
(43) Pub. Date: Aug. 13, 2026

(54) METHODS OF DYE CONJUGATION FOR ENHANCED FLUORESCENCE IMAGING

(71) Applicant: GMD12, LLC, Sarasota, FL (US)

(72) Inventor: Brian T. Bennett, Sarasota, FL (US)

(21) Appl. No.: 19/537,873

(22) Filed: Feb. 12, 2026

Related U.S. Application Data
(60) Provisional application No. 63/757,483, filed on Feb. 12, 2025.

(51) Int. Cl. G01N 33/533; G01N 21/64; G01N 33/58; G01N 33/68

(52) U.S. Cl. CPC G01N 33/533; G01N 21/6458; G01N 33/582; G01N 33/6854

(57) ABSTRACT

The invention discloses methods for generating antibody-fluorophore conjugates with enhanced fluorescence performance for analytical and imaging applications. Antibodies are conjugated to fluorescent dyes in an alkaline borate buffer and purified using gentle, membrane-based size-exclusion within conical tubes at reduced centrifugal force, minimizing fragmentation and removal of free dye. Controlled alkaline conditions and reduced dye-to-antibody ratios increase functional dye loading while preserving antibody integrity. The resulting conjugates demonstrate higher photon efficiency, reduced background fluorescence, and improved photostability compared to conventional preparations. These properties enable robust signal generation under substantially reduced excitation power across Widefield Fluorescence Microscopy, Widefield Structured Illumination Microscopy (SIM), Confocal Fluorescence Microscopy, super resolution Airyscan Fluorescence Microscopy, Lattice SIM, Lattice SIM2, and Stochastic Optical Reconstruction Microscopy modalities.

### CROSS-REFERENCE TO RELATED APPLICATION

The present application claims priority to U.S. Provisional Patent Application No. 63/757,483 filed Feb. 12, 2025, which is incorporated herein by reference in its entirety.

### TECHNICAL FIELD AND BACKGROUND

The present invention relates generally to methods for preparing antibody-fluorophore conjugates for fluorescence-based detection and imaging applications. The methods are applicable to immunoglobulin formats including IgG and F(ab′)₂, derived from any suitable species and directed against targets from any host organism. By way of non-limiting example, antibodies may be produced in species including goat, rabbit, mouse, rat, or other suitable hosts and may be directed against endogenous or exogenous proteins, including human proteins.

In particular, the invention relates to methods comprising chemically conjugating one or more fluorescent dyes to an antibody under controlled reaction conditions selected to increase functional dye loading while preserving antigen-binding specificity and functional integrity of the antibody. The resulting antibody-fluorophore conjugates are configured to generate detectable fluorescence signal under reduced excitation power relative to conventional conjugates in fluorescence-based analytical platforms, including flow cytometry and fluorescence microscopy, encompassing super-resolution imaging modalities.

Conventional antibody-dye conjugation workflows commonly employ standard buffer systems, acidic or poorly controlled pH conditions, and column-based purification techniques that may promote antibody fragmentation, incomplete dye coupling, or retention of unconjugated fluorescent species, resulting in elevated non-specific background signal and reduced quantitative accuracy and spatial confidence. Antibody-fluorophore conjugates prepared in accordance with the disclosed methods exhibit decreased nonspecific background fluorescence arising from unconjugated dye and antibody fragments, enabling improved signal discrimination, increased measurement fidelity, and enhanced optical detection accuracy.

High-parameter flow cytometry instruments, cell sorters, and super-resolution fluorescence microscopy platforms are conventionally designed to operate under high-excitation power conditions. These architectures typically employ laser sources in the tens-to-hundreds of milliwatt range at approximately 405 nm, 488 nm, 561 nm, 640 nm, and NIR wavelengths including approximately 750 nm, in addition to high-intensity broadband LED illumination. Super-resolution and computational imaging modalities-including patterned illumination and reconstruction-based approaches-rely on such elevated excitation power to maintain photon budgets sufficient for nanoscale structural resolution.

This reliance on high excitation power (i.e., the intensity of the light source) reflects a prevailing assumption that increased photon flux directly improves signal-to-noise ratio and spatial resolution. However, elevated excitation intensity introduces multiple mechanisms that reduce measurement confidence and effective resolution rather than improving it. High-power illumination accelerates photobleaching (i.e., degradation of the fluorophore leading to a change in its structure so that it can no longer fluoresce), induces photochemical damage, and increases nonspecific and endogenous fluorescence (endogenous substances produce fluorescence upon excitation), particularly in complex biological samples. These effects elevate background signal and temporal signal instability, thereby reducing contrast and impairing quantitative reproducibility.

At both visible and near-infrared excitation wavelengths, excessive excitation power further drives nonlinear fluorophore behavior (i.e., fluorescence emission is not directly proportional to excitation intensity), detector saturation (i.e., too many photons through the spectrometer), and optical cross-talk (i.e., leakage of light from one optical channel to another), leading to apparent signal amplification that is decoupled from true molecular abundance or localization. In reconstruction-based or patterned illumination systems, high excitation intensity exacerbates algorithmic artifacts (such as unintended, artificial structures, distortions, or enhancements), spatial distortion (i.e., warping of the image shape and spatial relationship between figures), and over-segmentation (wherein the object is incorrectly split into multiple, smaller, and distinct segments), resulting in images that exhibit nominally higher brightness but lower biological confidence.

Accordingly, systems optimized for high excitation power may exhibit reduced effective resolution (i.e., the reduced ability to distinguish closely spaced details), as the apparent spatial detail is dominated by power-induced fluorescence, optical artifacts, or reconstruction bias rather than true biological structure. These effects are particularly detrimental in applications requiring high confidence localization, quantitative comparison, or clinical-grade reproducibility, where elevated excitation power can produce visually compelling but biologically misleading results.

The disclosed methods address these limitations by enabling increased dye-to-antibody loading (i.e., the number of fluorophore molecules conjugated to a single antibody) while preserving antigen-binding specificity and functional integrity of the antibody. The resulting antibody-fluorophore conjugates exhibit improved photon efficiency, allowing robust fluorescence signal generation under substantially reduced excitation power relative to conventional conjugates. By reducing reliance on high excitation intensity, the disclosed methods mitigate power-induced photobleaching, nonspecific and endogenous fluorescence, nonlinear fluorophore behavior, and reconstruction-related artifacts that otherwise degrade measurement confidence. These improvements support enhanced detection sensitivity, quantitative reproducibility, and biological fidelity across fluorescence-based analytical and imaging applications, including high-throughput measurements, conventional microscopy, and super-resolution and single-molecule localization modalities.

### SUMMARY

According to one or more exemplary embodiments, a method for preparing an antibody-fluorophore conjugate is provided. The method may comprise equilibrating an antibody solution and a reactive fluorescent dye to ambient temperature, for example about 22±3° C. The antibody may be buffered into a borate buffer by combining an antibody-containing solution with a borate buffer and performing a first buffer exchange using a membrane-based centrifugal filtration device disposed within a centrifuge tube. The first buffer exchange may be conducted at a centrifugal force of about 500×g to about 3,000×g.

The method may further comprise determining an antibody concentration by ultraviolet-visible (UV-Vis) spectrophotometry, optionally at a wavelength including approximately 280 nm, and calculating an amount of fluorescent dye to be added based on the measured antibody concentration. The fluorescent dye may be introduced into the buffered antibody solution at a molar ratio of antibody to dye of about 3:1 to about 10:1 to form a conjugation mixture.

The conjugation mixture may be incubated at a selected temperature under agitation for a predetermined period, for example between about 1 hour and about 3 hours, thereby facilitating covalent coupling between the fluorescent dye and nucleophilic amino groups present on the antibody to generate an antibody-fluorophore conjugate. Residual unreacted dye may be quenched using a quenching agent under non-acidic conditions.

The method may further comprise purifying the antibody-fluorophore conjugate by sequential membrane-based purification cycles performed using a second membrane-based centrifugal filtration device disposed within a conical centrifuge tube. Each purification cycle may be performed at a neutral to mildly alkaline pH and at a centrifugal force of about 500×g to about 3,000×g. A purified antibody-fluorophore conjugate may be recovered following purification. The purified conjugate may subsequently be formulated in a storage buffer, wherein one or more parameters including concentration, volume, and pH are adjusted to produce a stabilized antibody-fluorophore conjugate composition.

According to an aspect of one or more example embodiments, there is provided a method for antibody-dye conjugation. The method may include equilibrating an antibody and a fluorescent dye to ambient temperature that may be 22±3° C. The methods may also include combining an antibody-containing solution with a borate buffer solution in a single container and subsequently performing a first buffer-exchange of the antibody into the borate buffer using a first membrane-based filtration device positioned within a first centrifuge tube. The first buffer exchange may be performed at a case-specific centrifugal force between 500×g and 3,000×g that depends on the antibody or antibody fragment, the dye chemistry hydrophobicity (e.g., whether and to what degree the dye is hydrophobic or hydrophilic), illumination wavelength.

The methods may also include determining an antibody concentration by ultraviolet-visible spectrophotometry using a range of wavelengths that may include 280 nm and calculating a dye input amount. The methods may also include adding the fluorescent dye to the buffered antibody solution in an amount that produces an antibody-to-dye molar ratio that may be between 3:1 and 10:1 and mixing the antibody and the fluorescent dye to form a conjugation mixture.

The methods may also include incubating the conjugation mixture by maintaining it at a selected temperature and under agitation for a predetermined period that may be between 1 and 3 hours. During this time, covalent bonds may form between the fluorescent dye and amino groups on the antibody to create an antibody-dye conjugate. The methods may also include quenching residual unreacted fluorescent dye using a quenching agent under non-acidic conditions. The methods may also include purifying the antibody-dye conjugate by performing sequential membrane-based size-exclusion purification cycles in a conical centrifuge tube. Each cycle may be carried out under neutral to mildly alkaline pH conditions at a centrifugal force that may be between 500×g and 3,000×g. The methods may also include recovering a purified fluorescent antibody-dye conjugate after purifying the antibody-dye conjugate. The methods may also include formulating the purified fluorescent antibody-dye conjugate. During formulation, the purified fluorescent antibody-dye conjugate may be added to a storage buffer and a concentration, volume, pH, or any combination thereof of the purified fluorescent antibody-dye conjugate is adjusted.

According to certain embodiments, the borate buffer may have a pH between 8.7 and 9.2. Additionally, according to certain embodiments, the borate buffer may have a pH between 8.9 and 9.1.

According to certain embodiments, the antibody-to-dye molar ratio may be between 10:1.

According to certain embodiments, the centrifugal force may be between 1,000×g and 2,500×g.

According to certain embodiments, the first and second centrifuge tube may be a conical centrifuge tube.

According to certain embodiments, the first and second membrane-based filtration device may have a molecular weight cutoff of 30 kilodaltons.

According to certain embodiments, the method may also include characterizing the purified fluorescent antibody-dye conjugate by ultraviolet-visible spectrophotometry to determine concentration and dye-to-antibody ratio.

According to certain embodiments, the method may also include applying the purified fluorescent antibody-dye conjugate to a biological sample. The biological sample may be sequentially imaged using a plurality of microscopy modalities which may include Widefield, Widefield Structured Illumination Microscopy (“SIM”), Confocal microscopy, super resolution Airyscan microscopy, lattice SIM, lattice SIM2, and STORM microscopy. Additionally, the biological sample may be imaged by illuminating the purified fluorescent antibody-dye conjugate with a light source that may include, but is not limited to, a broadband or multiband light-emitting diode (“LED”) excitation source, a wavelength-specific laser excitation, or a combination thereof.

According to an aspect of one or more exemplary embodiments, there is provided a method for antibody-dye conjugation. The method may include equilibrating a goat anti-mouse IgG antibody and a fluorescent dye to a ambient temperature. The ambient temperature may be 22±3° C. The method may also include placing the goat anti-mouse IgG antibody in a borate buffer which may have a pH between 8.7 and 9.2. The method may also include performing a buffer-exchange of the goat anti-mouse IgG antibody into the borate buffer using a membrane-based filtration device positioned within a conical centrifuge tube. The method may also include buffering the goat anti-mouse IgG antibody in a borate buffer by combining an antibody-containing solution with a borate buffer solution having a pH that may be between 8.7 and 9.2 in a single container to adjust the goat anti-mouse IgG antibody to an alkaline borate buffering environment, and may subsequently include performing a first buffer-exchange of the goat anti-mouse IgG antibody into the borate buffer using a first membrane-based filtration device positioned within a first conical centrifuge tube.

The first buffer-exchange may be performed at a centrifugal force between 1,000×g and 2,500×g. The method may also include determining a concentration of the goat anti-mouse IgG antibody by ultraviolet-visible spectrophotometry at a wavelength range that may include 280 nm and calculating a dye input amount. The method may also include adding the fluorescent dye to the buffered goat anti-mouse IgG antibody solution in an amount that produces an antibody-to-dye molar ratio that may be between 3:1 and 5:1 and mixing the goat anti-mouse IgG antibody and the fluorescent dye to form a conjugation reaction mixture. The method may also include incubating the conjugation reaction mixture by maintaining it at the ambient temperature and under gentle agitation for a predetermined period which may be between 1 and 3 hours. During this step, covalent bonds may form between the fluorescent dye and amino groups on the goat anti-mouse IgG antibody to create an antibody-dye conjugate. The method may also include quenching residual unreacted fluorescent dye using a quenching agent under non-acidic conditions. The method may also include purifying the antibody-dye conjugate by performing sequential membrane-based size-exclusion purification cycles using a second membrane-based filtration device positioned within a second conical centrifuge tube. Each cycle may be carried out under alkaline pH conditions at a centrifugal force that may be between 1,000×g and 2,500×g. The method may also include recovering a purified fluorescent antibody-dye conjugate after purifying the antibody-dye conjugate. The method may also include formulating the purified fluorescent antibody-dye conjugate, wherein the purified fluorescent antibody-dye conjugate may be added to a storage buffer, and a concentration, volume, pH, or any combination thereof of the purified fluorescent antibody-dye conjugate may be adjusted. The method may also include analyzing the purified fluorescent antibody-dye conjugate by ultraviolet-visible spectrophotometry to determine antibody concentration and dye-to-antibody ratio.

According to certain embodiments, the method may include applying the purified fluorescent antibody-dye conjugate to a biological sample and imaging the biological sample by fluorescence microscopy.

According to certain embodiments, the method may include sequentially imaging the biological sample using a plurality of microscopy modalities which may include Widefield Fluorescence Microscopy, Widefield SIM, Confocal Fluorescence Microscopy, super resolution Airyscan Fluorescence Microscopy, Lattice SIM and SIM2, and single-molecule Stochastic Optical Reconstruction Microscopy (“STORM”).

The sequential imaging is part of a walk down resolution workflow that serves as a performance threshold framework for conjugation methods described in this application. The use of sequential imaging modalities applies a threshold test point at each modality that evaluates the dye-conjugation chemistry with increasing analytical stringency to validate that the chemistry produces measurable structural outcomes across microscopy modalities. The resolution walkdown workflow may begin with a diffraction-limited microscope modality, proceed to a super-resolution microscope modality, and ultimately proceed to a single molecule resolution modality (i.e., STORM). Resolution walkdown techniques are discussed in more detail in the copending patent application Ser. No. 19/289,452, which is incorporated herein by reference in its entirety.

According to certain embodiments, the diffraction-limited microscope modality may be Widefield Fluorescence microscopy, Widefield Structured Illumination microscopy, and Confocal microscopy. According to certain embodiments, the super-resolution microscope modality may be Airyscan, lattice SIM, and lattice SIM2. According to certain embodiments, the single molecule microscope modality may be STORM.

According to certain embodiments, the resolution walkdown workflow may include sequentially performing Widefield imaging, Confocal imaging, Airyscan imaging, and STORM.

According to an aspect of one or more exemplary embodiments, there is provided a method for antibody-dye conjugation. The method may include equilibrating one or more fluorescent dyes to a ambient temperature that may be 22±3° C. The one or more fluorescent dyes may be configured for amine-reactive conjugation under alkaline conditions. The method may also include equilibrating one or more antibody reagents to the ambient temperature. The one or more antibody reagents may be configured for conjugation under alkaline borate conditions. The method may also include preparing a borate buffer that may have a pH between 8.7 and 9.2. The method may also include performing a buffer-exchange of the one or more antibody reagents into the borate buffer using a membrane-based filtration device positioned within a conical centrifuge tube. The membrane-based filtration device may include a molecular-weight cutoff membrane of 30 kilodaltons. The method may also include determining a concentration of the one or more antibody reagents by spectrophotometric analysis and calculating a reduced dye-input amount to achieve a desired dye-to-antibody ratio. The method may also include combining the one or more antibody reagents and one or more fluorescent dyes in the borate buffer and incubating the mixture to form an antibody-dye conjugate. The method may also include quenching residual reactive dye using a non-acidic quenching agent. The method may also include purifying the antibody-dye conjugate by a membrane-based size-exclusion purification performed under alkaline pH to form a purified antibody-fluorophore conjugate. The purification may include reduced centrifugal force, gravitational separation, or any combination thereof. The method may also include formulating the purified fluorescent antibody-dye conjugate, wherein the purified fluorescent antibody-dye conjugate is added to a storage buffer and a concentration, volume, pH, or any combination thereof of the purified fluorescent antibody-dye conjugate is adjusted.

The method may also include characterizing the formulated conjugate by spectrophotometric analysis to determine antibody concentration and dye-to-antibody ratio. The method may also include applying the formulated antibody conjugate to a biological sample and sequentially imaging the biological sample using a plurality of microscopy modalities which may include Confocal microscopy, Airyscan microscopy, SIM, SIM2, lattice SIM, lattice SIM2, and STORM.

Further features and advantages, as well as the structure and operation of various aspects, are described in detail below with reference to the accompanying drawings. It is noted that the specific aspects described herein are not intended to be limiting. Such aspects are presented herein for illustrative purposes only. Additional aspects will be apparent to persons skilled in the relevant art(s) based on the teachings contained herein.

### BRIEF DESCRIPTION OF THE FIGURES

Features, aspects, and advantages of the present invention are better understood when the following detailed description of the invention is read with reference to the accompanying figures, in which:

FIG. 1A is a schematic of an exemplary conjugation workflow, including buffer exchange, conjugation, quenching, and iterative purification steps.

FIG. 1B is a schematic diagram illustrating a representative, non-limiting conjugation workflow.

FIG. 2A presents flow cytometry data generated using antibody-fluorophore conjugates prepared in accordance with the disclosed methods, comparing a BRCA1 specific antibody conjugate with commercially available comparator conjugates and corresponding isotype controls.

FIG. 2B graph depicts a bar graph summarizing the mean fluorescence intensity for different staining conditions.

FIG. 3 presents representative flow cytometry graphical analysis selected from a set of ninety-six (96) secondary antibody conjugates generated using the disclosed methods, including both IgG and F(ab′)₂ formats. All corresponding flow cytometry data for the full set of tested conjugates are available as supporting documentation.

FIG. 4 provides fluorescence intensity line profiles illustrating signal differences across thousands of intracellular pixels within individual cells, demonstrating sensitivity for detecting positive intracellular reactions and resolving protein localization under finite photon collection conditions.

FIGS. 5A-5F illustrate a comparative Confocal microscopy analysis of an anti-human BRCA1 Super-Resolution Microscopy (“SRM”) Alexa Fluor 647 direct antibody-fluorophore conjugate prepared according to the methods described herein relative to a competitor direct conjugate, evaluated under DNA damage and control conditions using identical imaging parameters.

FIG. 5A shows competitor direct antibody-fluorophore conjugate prepared cells treated with a DNA-damaging agent (+2 μM etoposide) imaged using Confocal microscopy.

FIG. 5B shows Identifyn direct antibody-fluorophore conjugate prepared cells treated with a DNA-damaging agent (+2 μM etoposide) imaged using Confocal microscopy.

FIG. 5C shows competitor direct antibody-fluorophore conjugate prepared untreated cells imaged using Confocal microscopy.

FIG. 5D shows Identifyn direct antibody-fluorophore conjugate prepared untreated cells imaged using Confocal microscopy.

FIG. 5E shows an analysis of the line profile measurements across the nucleus and corresponding fluorescent signal of the competitor direct antibody-fluorophore conjugate.

FIG. 5F shows an analysis of the line profile measurements across the nucleus and corresponding fluorescent signal of the Identifyn direct antibody-fluorophore conjugate.

### DETAILED DESCRIPTION

The present invention will now be described more fully hereinafter with reference to the accompanying figures in which example embodiments of the invention are shown. However, the invention may be embodied in many different forms and should not be construed as limited to the representative embodiments set forth herein. Example embodiments are provided so that this disclosure will be thorough and complete and will fully convey the scope of the invention and enable one of ordinary skill in the art to make, use, and practice the invention.

Relative terms such as lower or bottom; upper or top; upward, outward, or downward; forward or backward; and vertical or horizontal may be used herein to describe one element's relationship to another element illustrated in the figures. It will be understood that relative terms are intended to encompass different orientations in addition to the orientation depicted in the drawings. Relative terminology such as “substantially” or “about” describes the specified materials, steps, parameters, or ranges as well as those that do not materially affect the basic and novel characteristics of the claimed inventions as a whole.

The disclosed processes improve dye loading efficiency and overall conjugate brightness by employing controlled reaction parameters, including specified antibody concentrations, controlled dye concentrations within the reaction volume, defined buffer compositions, predetermined quenching conditions, and repeated removal of unconjugated dye species. These elements enable efficient coupling of fluorophores (i.e., fluorescent chemical compounds) to antibody molecules while minimizing aggregation and non-productive side reactions. The improved optical performance is advantageous for fluorescence microscopy (including conventional and super-resolution modalities) and flow cytometry (i.e., a technique used to detect and measure the physical and chemical characteristics of a population of cells or particles), where photon output and stability directly impact sensitivity and quantitative accuracy.

In certain embodiments, increased functional dye loading results in part from the use of a borate buffer maintained within a pH range of about 8.7 to about 9.2 (e.g., about pH 9.0). In contrast, conventional methods commonly employ carbonate or bicarbonate buffers at pH values typically in the range of about 7.0 to about 8.3, in other words more acidic buffers. The present invention has discovered that operation in an alkaline borate buffer can preserve antibody functionality while increasing nucleophilicity of reactive amino groups, thereby promoting efficient covalent attachment of amine-reactive fluorescent dyes and improving batch-to-batch consistency.

The invention further incorporates membrane-based, size-exclusion purification configured for gentle separation of antibody-fluorophore conjugates from low-molecular-weight contaminants. In certain embodiments, a size-exclusion membrane is positioned within a conical centrifuge tube (e.g., 15 mL or 50 mL) and functions as a molecular-weight cutoff barrier that retains antibody-fluorophore conjugates while permitting passage of unconjugated dye, salts, and other low-molecular-weight species into the filtrate. In some embodiments, the membrane cutoff is selected to retain species above approximately 30 kilodaltons (“kDa”), although other cutoff values may be used depending on antibody format and dye chemistry.

In contrast to conventional ultrafiltration or column-based workflows that rely on high centrifugal forces and/or harsh acidic exposure, the disclosed methods perform purification within neutral to mildly alkaline pH ranges (e.g., about pH 7.0 to about pH 9.0). Separation may be achieved using stepwise membrane-based size exclusion in combination with controlled centrifugal or gravitational forces applied at reduced speeds sufficient to drive filtration while minimizing mechanical stress to the conjugates. Membrane cutoff selection, separation sequencing, and applied forces may be tailored according to antibody format, antigen specificity, and fluorescent dye properties.

Traditional membrane-based buffer exchange and concentration protocols commonly utilize centrifugal forces on the order of several thousand times gravity (“g”) (e.g., about 5,000×g to about 8,000×g). In contrast, the disclosed methods may perform membrane-based purification using reduced centrifugal forces, generally within a range of about 500×g to about 3,000×g (e.g., about 1,000×g to about 2,500×g), sufficient to drive separation while preserving conjugate integrity.

The combination of conical tube geometry and reduced centrifugal force can minimize shear-induced denaturation and aggregation, reduce foaming and air-liquid interface stress, and facilitate efficient recovery with reduced dead volume. Together with controlled alkaline conjugation and neutral-to-mildly alkaline purification, these features support uniform dye loading, reduced background fluorescence, and enhanced optical measurement precision.

Furthermore, the disclosed methods may employ reduced dye-to-antibody molar ratios relative to conventional near-equimolar protocols, for example by approximately two- to three-fold, to mitigate hydrophobic dye self-association, non-productive adsorption, and aggregation. Operating within reduced dye concentration ranges, in combination with controlled alkaline reaction conditions and gentle membrane-based purification, supports improved conjugate homogeneity, reduced background fluorescence, and enhanced optical measurement precision.

The disclosed methods address a longstanding challenge in antibody-fluorophore conjugation: increasing functional dye loading without compromising antibody binding activity, structural integrity, or optical performance. The ability to achieve robust fluorescence signal under reduced excitation power while preserving biological fidelity and reproducibility provides an unexpected technical advance over conventional workflows.

### Methods and Parameters

This section describes representative methods and parameters for preparation, conjugation, purification, and characterization of dye-antibody conjugates. The procedures are designed to support consistency and reproducibility by controlling reagent handling, buffer preparation, antibody processing, and reaction conditions. Documentation of calculations, reagent traceability, and critical timestamps may be used to support quality control.

In certain embodiments, parameters that distinguish the disclosed methods from conventional protocols include: (i) use of an alkaline borate buffer within a pH range of about 8.7 to about 9.2, and in certain embodiments about pH 9.0; (ii) membrane-based size-exclusion purification in a conical tube geometry operated at reduced centrifugal force within a range of about 500×g to about 3,000×g, and in certain embodiments around 2,000×g; and (iii) reduced dye to-antibody molar ratios, such as a protein-to-dye ratio around 5:1 (or functionally similar ratios) relative to conventional near-equimolar dye input. These parameters may be integrated throughout the preparation, conjugation, and purification workflow.

### Representative Workflows (Non-Limiting)

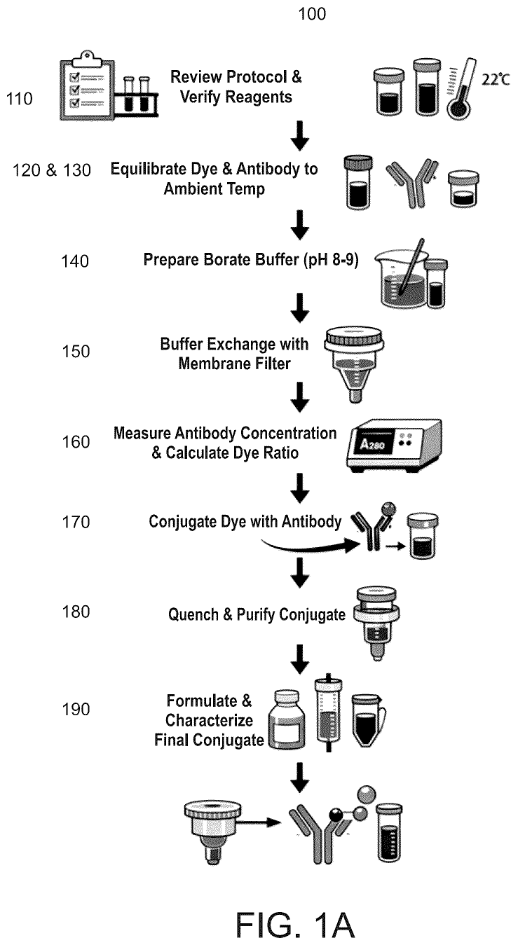

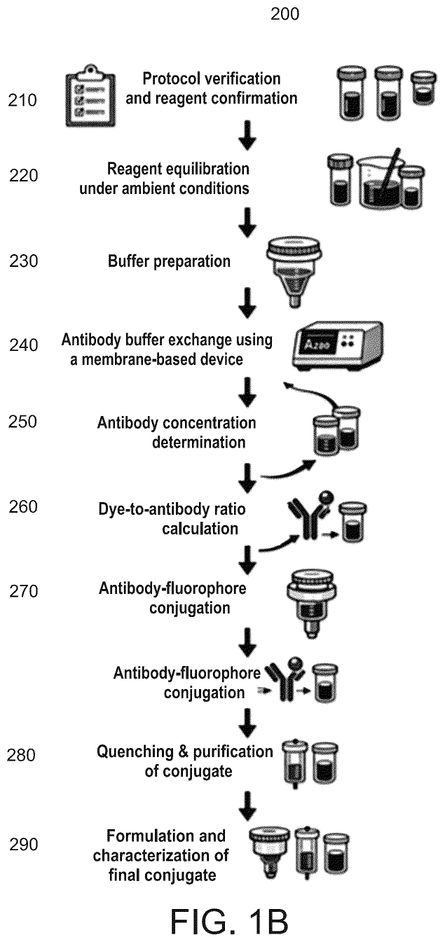

FIG. 1A is a schematic diagram illustrating a representative, non-limiting antibody-fluorophore conjugation workflow 100 according to some embodiments described herein.

As shown in FIG. 1A, the workflow 100 may begin with protocol verification and reagent confirmation 110, in which an operator retrieves a current conjugation protocol and verifies availability of required materials. Dye equilibration 120 may be performed by removing one or more fluorescent dye reagents from cold storage and allowing equilibration to ambient temperature (e.g., 22±3° C.). Antibody equilibration 130 may likewise be performed by equilibrating one or more antibody reagents to ambient temperature prior to processing. Times of removal and use may be documented.

A buffer preparation step 140 may include preparation of a borate-containing buffer (e.g., about 0.01 M), for example by dilution of a concentrated stock solution, and adjustment to an alkaline pH suitable for conjugation. The antibody may then undergo buffer exchange 150 into the prepared buffer using a membrane-based filtration device (e.g., 30 kDa MWCO), such as a molecular weight cutoff membrane, optionally under reduced centrifugal force to limit mechanical stress and preserve antibody integrity.

An analysis and calculation step 160 may include determination of antibody concentration, for example by ultraviolet-visible (“UV-Vis”) spectrophotometry at approximately 280 nm (A280), and calculation of a dye input amount selected to achieve a desired dye-to-antibody ratio between 3:1 and 10:1. A conjugation step 170 may then be initiated by combining the dye and antibody in the alkaline buffer and incubating under controlled conditions, such as gentle mixing for a defined reaction period (e.g., about 1-3 hours) and maintaining an incubation temperature at ambient temperature (e.g., 22±3° C. or room temperature).

Following conjugation, a quenching and purification step 180 may be performed in which residual reactive dye is quenched using a compatible quenching agent under non-acidic conditions, and the conjugate is purified, for example by stepwise membrane-based size-exclusion purification under neutral to mildly alkaline pH conditions. Quenching is a means to chemically stop the reaction by neutralizing all leftover dye so it does not attach to anything else (such as forming covalent bonds). During quenching, a quenching agent (e.g., Tris, glycine, ethanolamine) may be added to chemically react with and neutralize any residual reactive dye. Purification may be performed using reduced centrifugal force, gravitational separation, or combinations thereof. After quenching and purification, a purified fluorescent antibody-dye conjugate is recovered, meaning it is collected from the membrane-based filtration device. The purified fluorescent antibody-dye conjugate may be transferred to a clean storage tube or container.

The workflow 100 depicted in FIG. 1A may conclude with a formulation and characterization step 190 in which the purified conjugate is formulated in a physiologically compatible buffer and characterized, for example to determine concentration and dye-to-antibody ratio by spectrophotometric methods. During formulation of the purified conjugate, the conjugate is placed into a final storage or working buffer that is suitable for biological use. This may include adding the purified conjugate to a defined, physiologically compatible buffer (e.g., PBS, Tris buffered saline, HEPES buffer, and storage buffer containing stabilizers), adjusting, as required, the concentration, volume, or pH, and preparing the conjugate for storage, shipping, or biological use (e.g., microscopy, flow cytometry).

The steps illustrated in FIG. 1A are provided for explanatory purposes only. The order, inclusion, exclusion, repetition, or modification of steps may vary between embodiments, and the workflow 100 does not limit the scope of the claimed methods, which may be practiced with additional, fewer, or alternative steps, reagents, parameters, or conditions.

FIG. 1B is a schematic diagram illustrating a representative, non-limiting workflow 200 for preparing an antibody-fluorophore conjugate according to some embodiments described herein. As shown in FIG. 1B, the workflow may begin with protocol verification and reagent confirmation 210. One or more reagents may then undergo equilibration under ambient conditions 220 prior to further processing. A buffer preparation step 230 may be performed to generate a buffer suitable for conjugation.

The antibody may then undergo buffer exchange using a membrane-based device 240 to remove storage components or exchange into a conjugation-compatible buffer. Following buffer exchange, an antibody concentration determination step 250 may be performed, and a dye-to-antibody ratio calculation 260 may be carried out to determine an amount of fluorophore to be combined with the antibody.

An antibody-fluorophore conjugation step 270 may then be performed, resulting in formation of a labeled antibody. The conjugate may subsequently undergo quenching and purification 280 to remove unreacted fluorophore and reaction by-products. The workflow may conclude with formulation and characterization of the final conjugate 290.

The workflows illustrated in FIGS. 1A-1B are provided for explanatory purposes and does not require that the steps be performed in the order shown. One or more steps may be omitted, repeated, combined, or performed in a different sequence, and additional steps may be included, without departing from the scope of the claimed methods.

### Conjugated Antibodies Introduction

Two representative detection formats include (i) direct detection using a labeled primary antibody and (ii) indirect detection using a labeled secondary antibody that binds an unlabeled primary antibody. Indirect detection can increase local fluorophore density at the target site and, when combined with the disclosed conjugation chemistry, supports robust signal generation under reduced excitation power.

### Primary Conjugation Parameters (All Dyes)

Conjugated primary antibodies enable direct attachment of a detectable label to a primary antibody, supporting streamlined detection in applications including flow cytometry, immunohistochemistry, western blotting, and immunocytochemistry.

In certain embodiments, primary antibody concentration conditions may be evaluated during conjugation as set forth in Table 1.

TABLE 1
Primary Antibody Concentration Conditions

| | Primary Antibody | Dye Concentration in |
|---|---|---|
| Condition | Concentration (mg/mL) | Reaction Volume (mg/mL) |
| 1 | 4.0 | 0.8 |
| 2 | 3.0 | 0.6 |
| 3 | 2.0 | 0.4 |

In certain embodiments, conjugation is performed using a protein-to-dye ratio of approximately 5:1 (or a functionally similar ratio to produce the same effective dye loading) in a borate buffer maintained within the alkaline pH ranges described herein. Dye stock may be prepared immediately prior to use to support reproducibility.

Following the reaction, reactive dye may be quenched using a compatible quenching agent under non-acidic conditions. Unconjugated dye may be removed through sequential membrane-based purification cycles using a molecular-weight cutoff device (e.g., about 30 kDa MWCO) under reduced centrifugal force and/or gravitational separation, and the conjugate formulated in a physiologically compatible buffer (e.g., Phosphate-Buffered Saline or “PBS”).

### Secondary Conjugation Parameters (All Dyes)

Secondary antibody conjugates are configured to bind primary antibodies and are widely used in fluorescence-based analytical and imaging applications. In the context of the disclosed methods, fluorescent secondary conjugates provide particular advantages for low-excitation-power detection by recruiting multiple labeled secondaries to a single antigen-bound primary antibody, increasing local fluorophore density without requiring high excitation intensity.

Secondary antibody use may introduce additional incubation steps and can be limited by species specificity or non-specific interactions. The disclosed conjugation and purification methods mitigate background contributions by improving conjugate purity and stability.

In certain embodiments, secondary conjugation is conducted using IgG or F(ab′) 2 antibodies in an alkaline borate buffer within the pH ranges described herein, using reduced dye-to-antibody ratios, followed by gentle membrane-based purification under neutral to mildly alkaline pH conditions.

Following purification, conjugates may be characterized by UV-Vis spectrophotometry (via a UV-Vis spectrophotometer that measures the amount of ultraviolet and visible light that is absorbed by a sample) to determine antibody concentration and dye-to-antibody ratio, and stored under light-protected conditions at refrigerated temperature.

### Immunoglobulin G (“IgG”) Antibodies

IgG comprises multiple subclasses that differ in hinge length, flexibility, and disulfide bond composition. These subclass-dependent structural differences can influence solvent accessibility of reactive functional groups, aggregation propensity, and retention characteristics during purification. Accordingly, the disclosed conjugation and purification methods are adaptable across IgG subclasses and species origins, enabling consistent preparation of antibody-fluorophore conjugates while preserving antigen-binding specificity and optical performance.

### F(ab′)₂ Antibodies

F(ab′)₂ fragments are antigen-binding fragments derived from full-length IgG, typically produced by controlled enzymatic cleavage (e.g., pepsin digestion) that removes the Fc region while preserving bivalent antigen binding via the hinge region. F(ab′)₂ fragments generally exhibit a reduced molecular weight relative to intact IgG, on the order of approximately 100 kDa.

When prepared using the disclosed conjugation and purification methods, F(ab′)₂ fluorophore conjugates can exhibit preserved antigen-binding functionality, reduced background fluorescence, and consistent optical performance under reduced excitation power conditions.

Antibody fragments lacking the Fc region, including F(ab) and F(ab′)₂ formats, can facilitate improved penetration into dense samples and reduce Fc receptor-mediated background. These properties support use of Fc-deficient fragments in immunocytochemistry, immunohistochemistry, flow cytometry, and western blotting.

While monovalent F(ab) fragments can be advantageous for reduced steric bulk or sequential labeling, F(ab′)₂ fragments retain bivalent binding and may provide increased avidity. The disclosed methods are adaptable to both formats.

### Dye Conjugation—Working Examples

### Example One: Dye-Antibody Conjugation Using Goat Anti-Mouse F(Ab′)₂ and a Fluorescent Dye

The following example describes a representative procedure for conjugation of a fluorescent dye to a goat anti-mouse F(ab′)₂ antibody. This example is provided to illustrate one embodiment of the disclosed methods and is not intended to limit the scope of the invention.

Reagents are equilibrated to ambient temperature, the antibody is buffer-exchanged into an alkaline borate buffer, dye is combined at a reduced dye-to-antibody ratio, and the reaction is incubated under gentle mixing. Following conjugation, reactive dye is quenched under non-acidic conditions and unconjugated dye is removed by stepwise membrane-based purification under neutral to mildly alkaline pH conditions at reduced centrifugal force. Final conjugates are characterized by UV-Vis spectrophotometry to determine concentration and dye-to-antibody ratio and stored under light-protected conditions.

Conjugates prepared according to the disclosed methods may exhibit higher functional dye loading, improved photostability, reduced background fluorescence, and superior performance across conventional and super-resolution imaging modalities, including finite photon collection conditions.

The disclosed conjugates may be applied to biological samples for imaging by incubating the sample with the conjugate, washing, mounting, and imaging using microscopy configured for the dye excitation and emission characteristics. A resolution walkdown workflow may be applied to validate conjugate performance under increasing optical and analytical stringency.

### Example Two: Flow Cytometric Comparison of BRCA1 Direct Antibody

The following is an exemplary procedure for conjugation of a fluorescent dye to a Identifyn® BRCA1 Alexa Fluor 647 conjugated antibody. This example is provided to illustrate one embodiment of the disclosed methods and is not intended to limit the scope of the invention.

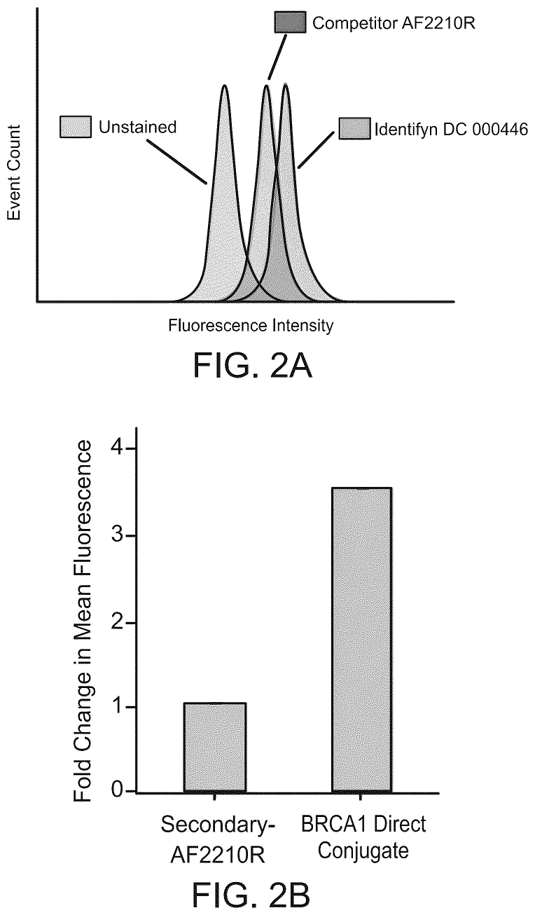

FIG. 2A shows a controlled comparison of how well an Identifyn® BRCA1 antibody and a commercial BRCA1 antibody detect BRCA1 inside cells using flow cytometry, using the same dye, same conditions, and proper controls. In FIG. 2A, flow cytometry analysis was performed using a Rabbit anti-Human BRCA1 recombinant monoclonal antibody directly conjugated to Identifyn® SRM Alexa Fluor 647 (IgG; BLR023N; Identifyn Cat. ID DC-000046). Performance was compared against a commercially available Human BRCA1 N-terminus Alexa Fluor 647-conjugated antibody (Novus Biologicals, Cat. ID AF2210R).

Methods. HeLa cells (human epithelial carcinoma cell line) were cultured according to standard protocols. To induce BRCA1 expression, cells were treated with 50 μM etoposide for 24 hours prior to sample preparation. Cells were then pelleted (i.e., collected) by centrifugation and fixed and permeabilized using the eBioscience FoxP3/Transcription Factor Staining Buffer Set (Invitrogen 00-5523-00) according to the manufacturer's instructions. This step is necessary because BRCA1 is inside the cell, not on the surface of the cell.

Following fixation and permeabilization, cells were washed twice and resuspended in Permeabilization/Wash Buffer (Invitrogen 00-8333-56) containing one of the following reagents: (i) 1 μg/mL Rabbit IgG Alexa Fluor 647 isotype control antibody (Invitrogen 51-4616-82), (ii) 1 μg/mL BRCA1 competitor Alexa Fluor647-conjugated antibody, or (iii) 1 μg/mL Identifyn® BRCA1 Alexa Fluor 647-conjugated antibody (DC-000046). Staining was performed for 2 hours at room temperature, protected from light. The first reagent Rabbit IgG Alexa Fluor 647 isotype control antibody is a negative control and should not specifically bind to BRCA1. The control antibody also shows background signal.

After staining, cells were washed twice and resuspended in Flow Cytometry Staining Buffer. Samples were transferred to FACS tubes, and 30,000 events per sample were acquired in the APC channel using a BD FACSymphonyS6 flow cytometer. In other words, after staining, the cells were washed and run on a flow cytometer. The instrument measured fluorescence in the APC channel, which detects Alexa Fluor 647. 30,000 cells were analyzed per sample to ensure reliable data.

Data Analysis. Flow cytometry data were analyzed using FlowJo software. Events were gated to exclude doublets (i.e., cells stuck together, which can distort results) prior to comparison of Allophycocyanin (“APC”) fluorescence intensity. Data were generated in triplicate (N=3) to confirm reproducibility.

Results. In FIG. 2A, the graph shows flow cytometry histograms comparing fluorescence intensity in the APC channel for three cell populations: (i) unstained cells; (ii) cells stained with a commercial BRCA1 antibody; and (iii) cells stained with the Identifyn® BRCA1 direct conjugate. The horizontal axis (x-axis) represents fluorescence intensity, while the vertical axis (y-axis) represents event count (number of cells). Through the histogram it can be observed that the unstained control appears as a narrow peak at low fluorescence, representing background signal. It can also be observed that both BRCA1-specific antibodies show a clear rightward shift relative to the unstained control, indicating specific BRCA1 detection. It can also be observed that the Identifyn® direct conjugate is shifted further to the right than the competitor antibody, indicating higher fluorescence intensity per cell. This suggests stronger signal and/or improved sensitivity of the Identifyn® conjugate under the same staining conditions.

In FIG. 2B, the graph depicts a bar graph summarizing the mean fluorescence intensity (“MFI”) for different staining conditions. The vertical axis represents fold change in mean fluorescence. The horizontal axis compares the competitor BRCA1 antibody and the Identifyn® BRCA1 direct conjugate. Through the graph, it can be observed that the competitor antibody produces approximately a 1-fold increase in fluorescence. It can also be observed that the Identifyn® BRCA1 direct conjugate produces a substantially higher fold increase (approximately 3-4-fold).

The use of particular reagents, consumables, and analytical platforms is described for clarity and convenience, and such references do not limit the invention, which may be practiced using additional or alternative materials, devices, or configurations.

### Aggregate Performance of Secondary Antibody Conjugates

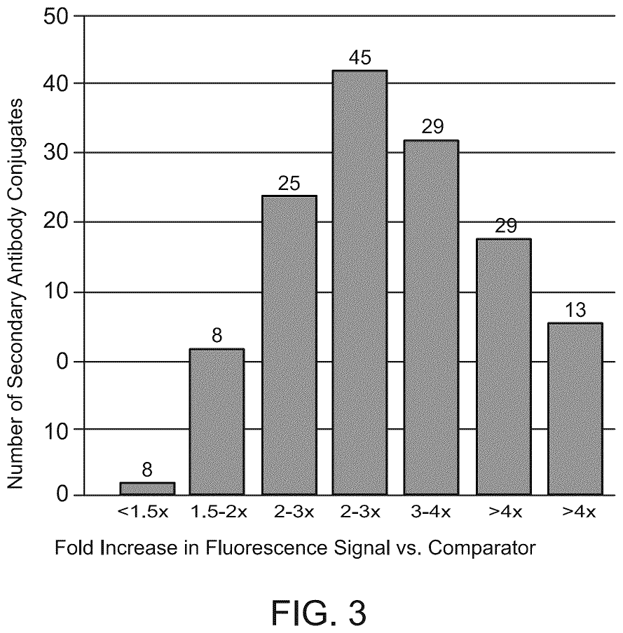

FIG. 3 depicts a histogram summarizing fluorescence signal improvement observed across a plurality of secondary antibody-fluorophore conjugates prepared according to the conjugation methods described herein.

As illustrated in FIG. 3, the histogram represents aggregated performance data from ninety-six (96) secondary antibody conjugates, including IgG and F(ab′)₂ formats derived from multiple host species and evaluated across multiple excitation wavelengths using flow cytometry. Each conjugate was assessed relative to a comparator or baseline conjugation condition under identical staining, acquisition, and analysis parameters.

The horizontal axis represents ranges of relative fluorescence signal improvement, expressed as fold-increase categories, while the vertical axis represents the number of conjugates exhibiting performance within each category. As shown, each evaluated conjugate exhibited an improvement in fluorescence signal relative to its comparator when prepared using the disclosed conjugation protocol, with a substantial proportion exhibiting multi-fold increases in signal intensity.

The observed improvements are attributable to the conjugation protocol described herein, which increases effective dye loading while reducing free dye, fragmented antibody species, and nonspecific background. As a result, photon efficiency and signal-to-background ratio are enhanced across a broad range of secondary antibody formats and detection channels.

FIG. 3 is provided to illustrate general performance trends across multiple antibody formats and does not require that any particular conjugate achieve a specific fold increase or absolute signal value. The number of conjugates, fold-increase ranges, antibody formats, fluorophores, and assay conditions may vary between embodiments without departing from the scope of the claimed methods.

### Microscopy—Conditions and Imaging

As used herein, the term “microscopy modalities” refers to the distinct optical imaging platforms, architectures, and operational techniques employed to visualize fluorescently labeled biological samples. Microscopy modalities differ in illumination source (e.g., LED-based or laser-based excitation), optical configuration, spatial resolution, detection strategy, and computational reconstruction requirements. These modalities span a hierarchy from diffraction-limited systems, such as widefield fluorescence microscopy and Confocal microscopy, to enhanced super resolution systems, such as Airyscan and SIM, and further to single molecule resolution platforms (i.e., STORM).

Each modality imposes different demands on fluorophore brightness, photostability, signal-to-background performance, and photon efficiency. Therefore, the fluorescence characteristics of the conjugates prepared according to the disclosed methods are evaluated across multiple microscopy modalities (i.e., the resolution walkdown method, see copending application Ser. No. 19/289,452 incorporated herein by reference) to validate the dye conjugation chemistry in step-wise fashion. Each modality of the walkdown provides a specific datapoint that is an independent pass/fail test point and where each datapoint is interdependent on the entirety of the walkdown method to support the conjugation methodology. The walk down method demonstrate the robustness of the dye chemistry under progressively increasing optical and analytical stringency.

This section describes microscopy conditions, imaging modalities, and acquisition parameters used to evaluate conjugate performance. Imaging was conducted across a hierarchical spectrum of platforms (a “resolution walkdown” or “resolution stepdown”) spanning conventional diffraction-limited microscopy, super-resolution microscopy, and single-molecule localization modalities.

Conventional platforms evaluated include widefield fluorescence microscopy, widefield structured illumination microscopy (also referred to as widefield SIM), and Confocal microscopy (diffraction-limited laterally, typically about 200-250 nm). Super-resolution modalities evaluated include Airyscan, lattice SIM, lattice SIM2, and single-molecule localization microscopy (such as STORM).

The imaging workflow is directly enabled by upstream conjugation and purification steps. Conjugation parameters translate into measurable imaging outcomes including brightness, background suppression, signal stability, and binding fidelity. Microscopy is employed as functional validation of conjugation chemistry under increasing optical and analytical stringency.

### Microscopy Modalities

Widefield Fluorescence Microscopy (LED-Based). Widefield fluorescence microscopy serves as the initial modality in the resolution walkdown, providing two-dimensional images across a broad field of view for rapid assessment of sample quality, labeling performance, and gross cellular organization. Widefield collects fluorescence from the full specimen thickness without optical sectioning, producing a two-dimensional projection of signal from multiple axial planes. Under typical conditions, lateral resolution is diffraction-limited (about 200-250 nm) and axial resolution is substantially lower. Additionally, out-of-focus fluorescence reduces contrast in thick or densely labeled samples. Within the disclosed workflows, widefield imaging functions as a biological and technical baseline prior to progression to higher-stringency modalities.

Widefield Structured Illumination Microscopy (LED-Based). Widefield SIM extends widefield imaging by introducing patterned illumination and computational combination of multiple phase images to improve contrast and extract additional spatial information while remaining fundamentally constrained by the diffraction limit. Widefield SIM improves rejection of out-of-focus fluorescence and enhances contrast in thin or moderately thick specimens, and is compatible with low-power broadband LED excitation. Because structured illumination requires acquisition of multiple patterned frames, signal stability and brightness under low excitation are critical. The disclosed conjugates support robust reconstruction at reduced LED drive currents, minimizing photobleaching and illumination-induced artifacts.

Confocal Fluorescence Microscopy (Laser-Based). Confocal microscopy follows widefield modalities and introduces optical sectioning by spatial filtering of emitted fluorescence through a pinhole. Lateral resolution remains diffraction-limited (about 200-250 nm), while axial resolution is improved via sectioning. Because only a fraction of emitted photons reaches the detector, Confocal imaging is demanding with respect to photon efficiency. The disclosed conjugates enable robust Confocal imaging under reduced excitation power relative to conventional conjugates.

Airyscan Fluorescence Microscopy (Laser-Based; Detector-Based Super-Resolution). Super resolution Airyscan microscopy builds upon Confocal optical sectioning by using a multi-element detector array and computational photon reassignment. Under typical conditions, Airyscan achieves lateral resolution of approximately 120-140 nm and axial resolution of approximately 350-400 nm, depending on configuration. Airyscan reconstruction is sensitive to background and photostability. The disclosed conjugates support stable reconstruction at reduced excitation power.

Lattice Structured Illumination Microscopy (Laser-Based Super-Resolution). Lattice SIM employs patterned lattice illumination and reconstruction to extend spatial resolution beyond the diffraction limit. Under typical conditions, lattice SIM provides lateral resolution of approximately 80-120 nm and axial resolution of approximately 250-300 nm, depending on wavelength, numerical aperture, and reconstruction parameters. Lattice SIM places increased demands on photon efficiency and background suppression. The disclosed conjugates support robust reconstruction under finite photon collection conditions at reduced excitation power.

Lattice Structured Illumination Microscopy Squared (Laser-Based). Lattice SIM2 is a higher-stringency extension of lattice SIM that employs nonlinear illumination and higher-order reconstruction to extract additional spatial frequency information. Under appropriate conditions, lattice SIM2 can achieve lateral resolution in the lower tens of nanometers, for example approximately 40-60 nm, with performance dependent on signal quality and reconstruction parameters. Because reconstruction relies on accurate recovery of weak high-frequency components, lattice SIM2 is exceptionally sensitive to photon budget, camera performance, and residual background from free dye or fragmented species. Successful performance at the lattice SIM2 level provides a stringent validation that conjugation chemistry and purification have passed a critical sensitivity threshold for progression to single-molecule localization.

Stochastic Optical Reconstruction Microscopy (STORM; Laser-Based Single-Molecule Localization). STORM relies on stochastic emission of sparse subsets of fluorophores and nanometer-scale localization of individual emission events. Under appropriate conditions, STORM achieves lateral localization precision of approximately 10-30 nm and axial localization precision of approximately 30-60 nm, depending on configuration, labeling density, and photon yield. STORM performance is fundamentally constrained by photon statistics and background suppression; residual free dye, nonspecific fluorescence, and heterogeneous labeling degrade localization precision and introduce false localizations. The disclosed conjugates provide photon efficiency, purity, and stability required for STORM under finite photon budgets, and lattice SIM2 success is predictive of STORM viability.

### LED-Based and Laser-Based Excitation Performance of Disclosed Antibody-Fluorophore Conjugates.

Unless explicitly stated otherwise, references herein to Confocal microscopy, Airyscan microscopy, SIM, SIM2, lattice SIM, lattice SIM2, and STORM refer to laser-based excitation systems. References to LED-based excitation systems are limited to widefield fluorescence microscopy and widefield SIM.

LED-based systems are commonly used in widefield microscopy and widefield SIM due to reduced complexity and cost. LED illumination typically provides lower peak spectral irradiance than laser excitation at comparable wavelengths, which can limit excitation efficiency for intracellular assays and increase dependence on near-maximum illumination output when conventional conjugates are used.

Antibody-fluorophore conjugates prepared according to the present disclosure were evaluated under broadband LED illumination using LED-based widefield and LED-based widefield SIM configurations. Illumination intensity was quantified as a percentage of maximum available LED output for each wavelength band.

Across an aggregated dataset comprising more than 1,000 samples acquired using LED-based widefield and LED-based widefield SIM modalities, the average LED power utilization required to achieve robust fluorescence signal is summarized in Table 2.

Table 2 represents the average broadband LED excitation power utilization for antibody-fluorophore conjugates prepared according to the methods described herein and evaluated using LED-based widefield and LED-based widefield SIM.

TABLE 2
Average Broadband LED Power Utilization -
Widefield and Widefield SIM

| Wavelength (nm) | Avg. LED Power (%) |
|---|---|
| 405 | 17.57 |
| 488 | 27.32 |
| 532 | 24.18 |
| 555 | 20.25 |
| 594 | 21.11 |
| 647 | 20.98 |
| 680 | 25.42 |
| 750 | 30.16 |

The values in Table 2 demonstrate that strong fluorescence signal can be achieved at LED drive levels substantially below maximum output, including for longer-wavelength dyes and intracellular protein targets. These results indicate that excitation efficiency is governed primarily by conjugate photon efficiency and background suppression rather than illumination hardware alone.

As shown in Table 2, the excitation performance is summarized across multiple wavelength bands, including representative excitation bands centered at approximately 405 nm, 488 nm, 532 nm, 555 nm, 594 nm, 647 nm, 680 nm, and 750 nm. For each wavelength band, an associated average LED drive level is shown, expressed as a percentage of the maximum available LED output for the corresponding illumination channel.

The values illustrated in Table 2 represent aggregated measurements obtained from a dataset comprising a plurality of samples, including intracellular targets, acquired using LED-based widefield and LED-based widefield SIM configurations. In the illustrated embodiment, robust fluorescence signal is achieved at LED excitation levels substantially below maximum output across all evaluated wavelength bands, including longer-wavelength excitation bands.

Table 2 demonstrates that antibody-fluorophore conjugates prepared according to the present disclosure exhibit high excitation efficiency under broadband LED illumination, reducing reliance on near-maximum illumination output. The observed performance indicates that fluorescence signal quality is governed primarily by conjugate photon efficiency and background suppression rather than illumination hardware output alone.

Although Table 2 is described with reference to LED-based widefield and widefield SIM modalities, the disclosed conjugates may additionally be evaluated under reduced excitation conditions in laser-based microscopy systems and flow cytometry platforms, where reduced excitation intensity mitigates photobleaching, minimizes illumination-induced artifacts, and preserves biological structure.

The data illustrated in Table 2 is provided for explanatory purposes only. The number of samples, excitation wavelengths, illumination modalities, and excitation power levels may vary between embodiments, and the figure does not limit the scope of the claimed methods or compositions.

Conjugates were further evaluated under reduced excitation power conditions in laser-based microscopy and flow cytometry, the results of which are shown below in Table 3. Reduced excitation intensity mitigates photobleaching and illumination-induced artifacts and preserves biological structure, particularly in samples requiring quantitative reproducibility.

TABLE 3
Average Laser Excitation Intensities - Airyscan and SIM/SIM2

| Wavelength (nm) | Airyscan (mW) | SIM/SIM2 (mW) |
|---|---|---|
| 405 | 0.09 | 2.55 |
| 488 | 0.39 | 1.94 |
| 532 | 1.05 | 3.05 |
| 555 | 0.53 | 2.16 |
| 594 | 0.80 | 1.33 |
| 647 | 0.68 | 0.07 |
| 680 | 2.02 | 3.51 |

Table 3 is a visual representation of the average laser excitation intensity for antibody-fluorophore conjugates prepared according to the methods described herein and evaluated using laser-based microscopy modalities, including super resolution Airyscan and structured illumination microscopy (SIM and SIM2).

As shown in Table 3, excitation performance is summarized across representative excitation wavelengths, including wavelengths centered at approximately 405 nm, 488 nm, 532 nm, 555 nm, 594 nm, 647 nm, and 680 nm. For each wavelength, corresponding average excitation intensities are shown for super resolution Airyscan and SIM/SIM2 imaging modalities, expressed in units of laser output power.

The values illustrated in Table 3 represent representative averages derived from aggregated datasets acquired across a plurality of samples and imaging configurations. In the illustrated embodiment, robust fluorescence signal is achieved at laser excitation intensities substantially below maximum available output for each wavelength and modality, including in high-resolution and super-resolution imaging contexts.

Table 3 demonstrates that antibody-fluorophore conjugates prepared according to the present disclosure enable reduced laser excitation requirements across multiple laser-based imaging modalities. Reduced excitation intensity mitigates photobleaching, limits illumination-induced artifacts, and preserves biological structure, particularly in applications requiring quantitative reproducibility or repeated imaging.

Although Table 3 is described with reference to super resolution Airyscan and SIM/SIM2 modalities, the disclosed conjugates may be employed in other laser-based imaging systems, including Confocal microscopy, lattice SIM, lattice SIM2, STORM, and flow cytometry platforms, under reduced excitation conditions relative to conventional conjugates.

The data illustrated in Table 3 is provided for explanatory purposes only. The number of samples, excitation wavelengths, laser powers, imaging modalities, and acquisition parameters may vary between embodiments, and Table 3 does not limit the scope of the claimed methods or compositions to specific excitation intensities or wavelength-dependent thresholds.

### Comparative Excitation Efficiency Across Illumination Modalities.

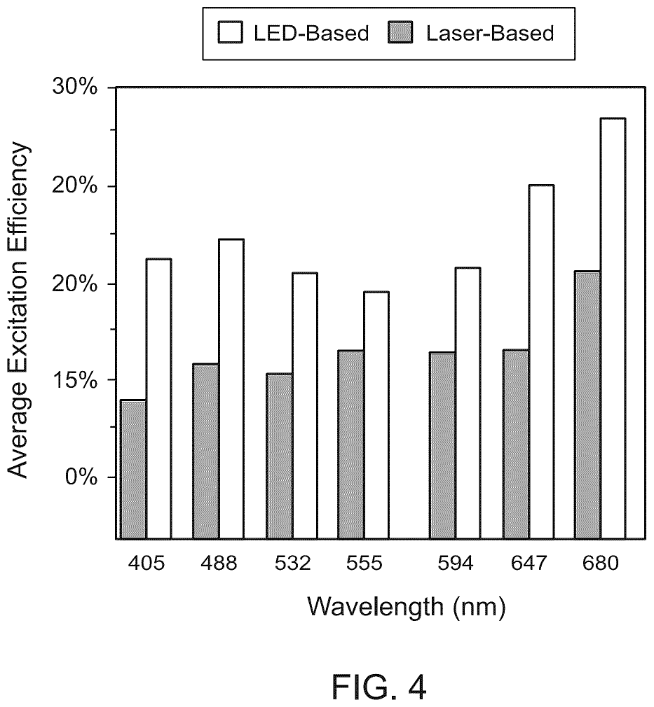

FIG. 4 compares Tables 2 and 3 to illustrate excitation efficiency characteristics of antibody-fluorophore conjugates prepared according to the present disclosure across both LED-based illumination systems and laser-based excitation systems. As shown in Table 2, the disclosed conjugates achieve robust fluorescence signal under broadband LED illumination at illumination levels substantially below maximum available output across multiple wavelength bands. As shown in Table 3, similarly a robust fluorescence signal is achieved in laser-based imaging modalities, including super resolution Airyscan and structured illumination microscopy (SIM and SIM2), at reduced laser excitation intensities.

The comparative data of FIG. 4 demonstrate that excitation efficiency is governed primarily by conjugate photon efficiency, target-specific labeling, and background suppression rather than illumination hardware alone. Across both LED- and laser-based modalities, the disclosed conjugates enable reduced excitation requirements, thereby mitigating photobleaching, limiting illumination-induced artifacts, and preserving biological structure.

The ability of the disclosed conjugates to maintain high signal quality under reduced excitation conditions across fundamentally different illumination architectures supports their applicability to a broad range of imaging modalities, including Widefield microscopy, Widefield SIM microscopy, Confocal microscopy, and super-resolution techniques that include super resolution Airyscan, SIM/SIM2, and single molecule microscopy (i.e., STORM) and flow cytometry, without reliance on high-power illumination hardware.

The comparative illustrations are provided for explanatory purposes only and do not limit the disclosed methods or compositions to specific illumination sources, excitation intensities, or imaging platforms.

### Comparative Confocal Microscopy Performance of Direct Conjugates

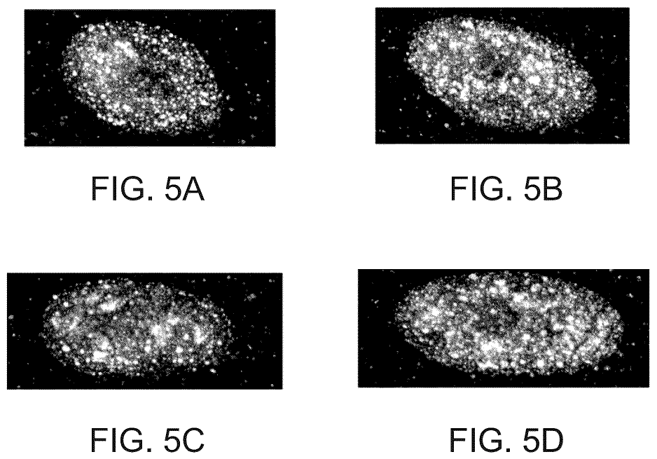

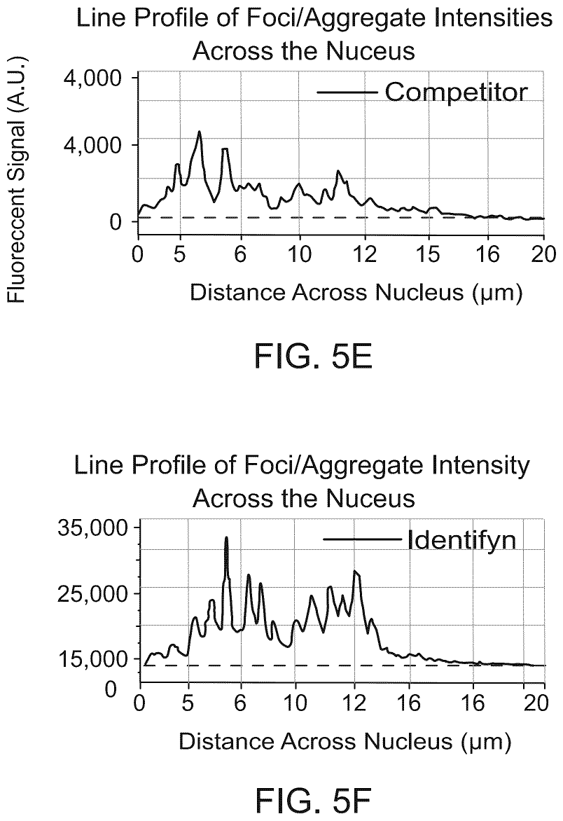

FIGS. 5A-5D present a comparative Confocal microscopy analysis of an anti-human BRCA1 SRM Alexa Fluor 647 direct antibody-fluorophore conjugate (i.e., the Identifyn direct conjugate) prepared according to the methods described herein, evaluated against a competitor direct conjugate under DNA-damage and control conditions using identical imaging parameters. FIGS. 5E-5F provide corresponding graphical line-profile analyses across the nucleus, showing quantitative fluorescence signal measured in arbitrary units (“A.U.”).

As shown in FIGS. 5A-5D, cells treated with a DNA-damaging agent (+2 μM etoposide) (see FIGS. 5A-5B) and untreated cells (see FIGS. 5C-5D) were imaged using Confocal microscopy under matched acquisition settings, including excitation wavelength, laser power, line step size, averaging, detector voltage, and detector gain. Under etoposide, both conjugates reveal BRCA1 nuclear foci; however, the Identifyn direct conjugate (see FIG. 5B) displays brighter and more numerous foci, indicating a stronger signal. Notably, excitation at approximately 647 nm for the Identifyn direct conjugate was achieved using low laser output-approximately 0.07% of available power-consistent with the efficiency enabled by the disclosed methods. Under no-etoposide conditions (see FIGS. 5C-5D), both conjugates show baseline staining, but the Identifyn direct conjugate (see FIG. 5D) again provides brighter, cleaner signal with reduced background. Collectively, the Identifyn direct conjugate exhibits substantially increased fluorescence intensity relative to the competitor across both conditions (compare FIGS. 5B, 5D, 5F to FIGS. 5A, 5C, 5E).

The competitor direct conjugate produced arithmetic mean intensities of 2,477 A.U. under etoposide and 2,096 A.U. under no etoposide. The Identifyn direct conjugate produced a mean intensity of 2,477 A.U. under etoposide and 2,459 A.U. under no etoposide.

In FIGS. 5E-5F, line-profile measurements across the nucleus demonstrate an approximately three- to four-fold increase in peak fluorescence intensity for the Identifyn direct conjugate relative to the competitor when imaging BRCA1 foci following DNA damage. The competitor direct conjugate's maximum signal was 3,288 A.U. with a mean of 2,477 A.U., whereas the Identifyn direct conjugate achieved a maximum of 28,591 A.U. with a mean of 2,076 A.U. These profiles reflect dramatically higher peak intensities and more distinct BRCA1 foci for the Identifyn direct conjugate.

In the presence of etoposide, the Identifyn direct conjugate reveals discrete, high-contrast BRCA1 nuclear foci and aggregate structures consistent with recruitment to DNA damage sites, while maintaining low diffuse background in untreated cells. The competitor direct conjugate shows reduced signal amplitude and diminished structural definition under the same low-power excitation conditions. The enhanced signal intensity and structural fidelity achieved by the Identifyn direct conjugate are attributable to the conjugation chemistry described herein, which increases functional dye loading while minimizing free dye, antibody fragmentation, and nonspecific background. This improves photon efficiency and enables robust Confocal imaging at reduced excitation power, mitigating photobleaching, phototoxicity, and illumination-induced artifacts.

Accordingly, FIGS. 5A-5F demonstrate that antibody-fluorophore conjugates prepared according to the present disclosure exhibit superior fluorescence performance relative to conventional direct conjugates, including multi-fold signal enhancement, improved signal-to-background ratio, and artifact-free structural imaging under reduced-excitation regimes.

The data shown in FIGS. 5A-5F are provided for illustrative purposes and do not limit the scope of the claimed methods or compositions. Imaging conditions, excitation power, fluorophore identity, and antibody targets may vary while maintaining the performance advantages described herein.

The comparative results illustrated in FIGS. 5A-5F are consistent with the conjugation workflow depicted in FIGS. 1A-1B and with the reduced-excitation efficiencies demonstrated in Table 2 (LED-based systems) and Table 3 (laser-based systems), collectively confirming that the disclosed conjugates enable multi-fold signal enhancement and high-fidelity imaging across diverse fluorescence modalities.

What is claimed is:

1. A method for antibody-dye conjugation, comprising the steps of:
(a) equilibrating an antibody and a fluorescent dye to ambient temperature;
(b) combining an antibody-containing solution with a borate buffer solution in a single container,
(c) performing a first buffer-exchange of the antibody into the borate buffer using a first membrane-based filtration device positioned within a first centrifuge tube, wherein the first buffer exchange is performed at a centrifugal force between 500×g and 3,000×g;
(d) determining an antibody concentration by ultraviolet-visible spectrophotometry using a range of wavelengths that include 280 nm and calculating a dye input amount;
(e) adding the fluorescent dye to the buffered antibody solution in an amount that produces an antibody-to-dye molar ratio between 3:1 and 10:1 and mixing the antibody and the fluorescent dye to form a conjugation mixture;
(f) incubating the conjugation mixture by maintaining it at a selected temperature and under agitation for a predetermined period between 1 and 3 hours, wherein covalent bonds form between the fluorescent dye and amino groups on the antibody to create an antibody-dye conjugate;
(g) quenching residual unreacted fluorescent dye using a quenching agent under non-acidic conditions;
(h) purifying the antibody-dye conjugate by performing sequential membrane-based size-exclusion purification cycles using a second membrane-based filtration device positioned within a second conical centrifuge tube, each cycle being carried out under neutral to mildly alkaline pH conditions at a centrifugal force between 500×g and 3,000×g;
(i) recovering a purified fluorescent antibody-dye conjugate after purifying the antibody-dye conjugate; and
(j) formulating the purified fluorescent antibody-dye conjugate, wherein the purified fluorescent antibody-dye conjugate is added to a storage buffer and a concentration, volume, pH, or any combination thereof of the purified fluorescent antibody-dye conjugate is adjusted.

2. The method of claim 1, wherein the borate buffer has a pH between 8.7 and 9.2.

3. The method of claim 2, wherein the borate buffer has a pH between 8.9 and 9.1.

4. The method of claim 1, wherein the antibody-to-dye molar ratio is 10:1.

5. The method of claim 1, wherein the centrifugal force is between 1,000×g and 2,500×g.

6. The method of claim 1, wherein the first centrifuge tube and the second centrifuge tube are conical.

7. The method of claim 1, wherein the first membrane-based filtration device and the second membrane-based filtration device have a molecular weight cutoff of 30 kilodaltons.

8. The method of claim 1, further comprising characterizing the purified fluorescent antibody-dye conjugate by ultraviolet-visible spectrophotometry to determine concentration and dye-to-antibody ratio.

9. The method of claim 1, further comprising applying the purified fluorescent antibody-dye conjugate to a biological sample.

10. The method of claim 9, further comprising sequentially imaging the biological sample using a plurality of microscopy modalities selected from a group comprising widefield microscopy, widefield structured illumination microscopy, Confocal microscopy, Airyscan microscopy, lattice structured illumination microscopy (SIM), lattice SIM2, and Stochastic Optical Reconstruction Microscopy.

11. The method of claim 9, further comprising the steps of imaging the biological sample by illuminating the purified fluorescent antibody-dye conjugate with a broadband or multiband light-emitting diode excitation source.

12. The method of claim 9, further comprising the steps of imaging the biological sample by illuminating the purified fluorescent antibody-dye conjugate with a light source that comprises a broadband or multiband light-emitting diode excitation source, a wavelength-specific laser excitation source, or a combination thereof.

13. A method for antibody-dye conjugation, comprising the steps of:
(a) equilibrating a goat anti-mouse IgG antibody and a fluorescent dye to a ambient temperature;
(b) placing the goat anti-mouse IgG antibody in a borate buffer having a pH between 8.7 and 9.2.9 by performing a buffer-exchange of the goat anti-mouse IgG antibody into the borate buffer using a membrane-based filtration device positioned within a conical centrifuge tube;
(c) buffering the goat anti-mouse IgG antibody in a borate buffer by combining an antibody-containing solution with a borate buffer solution having a pH between 8.7 and 9.2 in a single container to adjust the goat anti-mouse IgG antibody to an alkaline borate buffering environment, and subsequently performing a first buffer-exchange of the goat anti-mouse IgG antibody into the borate buffer using a first membrane-based filtration device positioned within a first conical centrifuge tube, wherein the first buffer-exchange is performed at a centrifugal force between 1,000×g and 2,500×g;
(d) determining a concentration of the goat anti-mouse IgG antibody by ultraviolet-visible spectrophotometry at a wavelength range that includes 280 nm and calculating a dye input amount;
(e) adding the fluorescent dye to the buffered goat anti-mouse IgG antibody solution in an amount that produces an antibody-to-dye molar ratio between 3:1 and 5:1 and mixing the goat anti-mouse IgG antibody and the fluorescent dye to form a conjugation reaction mixture;
(f) incubating the conjugation reaction mixture by maintaining it at the ambient temperature and under gentle agitation for a predetermined period between 1 and 3 hours, wherein covalent bonds form between the fluorescent dye and amino groups on the goat anti-mouse IgG antibody to create an antibody-dye conjugate;
(g) quenching residual unreacted fluorescent dye using a quenching agent under non-acidic conditions;
(h) purifying the antibody-dye conjugate by performing sequential membrane-based size-exclusion purification cycles using a second membrane-based filtration device positioned within a second conical centrifuge tube, each cycle being carried out under alkaline pH conditions at a centrifugal force between 1,000×g and 2,500×g;
(i) recovering a purified fluorescent antibody-dye conjugate after purifying the antibody-dye conjugate;
(j) formulating the purified fluorescent antibody-dye conjugate, wherein the purified fluorescent antibody-dye conjugate is added to a storage buffer and a concentration, volume, pH, or any combination thereof of the purified fluorescent antibody-dye conjugate is adjusted; and
(k) analyzing the purified fluorescent antibody-dye conjugate by ultraviolet-visible spectrophotometry to determine antibody concentration and dye-to-antibody ratio.

14. The method of claim 13, further comprising applying the purified fluorescent antibody-dye conjugate to a biological sample and imaging the biological sample by fluorescence microscopy.

15. The method of claim 14, wherein imaging the biological sample comprises sequentially imaging the biological sample using a plurality of microscopy modalities selected from widefield microscopy, widefield structured illumination microscopy, Confocal microscopy, Airyscan microscopy, lattice structured illumination microscopy (SIM), lattice SIM2, and Stochastic Optical Reconstruction Microscopy.

16. The method of claim 14, wherein imaging the biological sample comprises a resolution walkdown workflow beginning with a diffraction-limited microscope modality and proceeding to a super-resolution microscope modality and then proceeding to a single-molecule microscope modality.

17. The method of claim 16, wherein the diffraction-limited microscope modality is selected from the group comprising widefield fluorescence microscopy, widefield structured illumination microscopy, and Confocal microscopy.

18. The method of claim 16, wherein the super-resolution microscope modality is selected from a group comprising Airyscan, lattice SIM, and lattice SIM2.

19. The method of claim 16, wherein the resolution walkdown workflow comprises sequentially performing widefield imaging, widefield SIM imaging, Confocal imaging, Airyscan imaging, Lattice SIM imaging, Lattice SIM2 imaging, and STORM imaging.

20. A method for antibody-dye conjugation, comprising the steps of:
(a) equilibrating one or more fluorescent dyes to a ambient temperature, wherein the one or more fluorescent dyes are configured for amine-reactive conjugation under alkaline conditions;
(b) equilibrating one or more antibody reagents to the ambient temperature, the one or more antibody reagents are configured for conjugation under alkaline borate conditions;
(c) preparing a borate buffer having a pH between 8.7 and 9.2;
(d) combining the one or more antibody reagents into the borate buffer using a membrane-based filtration device positioned within a conical centrifuge tube, the membrane-based filtration device comprising a molecular-weight cutoff membrane of 30 kilodaltons;
(e) determining a concentration of the one or more antibody reagents by spectrophotometric analysis and calculating a reduced dye-input amount to achieve a desired dye-to-antibody ratio;
(f) combining the one or more antibody reagents and one or more fluorescent dyes in the borate buffer and incubating the mixture to form an antibody-dye conjugate;
(g) quenching residual reactive dye using a non-acidic quenching agent;
(h) purifying the antibody-dye conjugate by a membrane-based size-exclusion purification performed under alkaline pH to form a purified antibody-fluorophore conjugate, the purification comprising reduced centrifugal force, gravitational separation, or any combination thereof;
(i) formulating the purified fluorescent antibody-dye conjugate, wherein the purified fluorescent antibody-dye conjugate is added to a storage buffer and a concentration, volume, pH, or any combination thereof of the purified fluorescent antibody-dye conjugate is adjusted;
(j) characterizing the formulated conjugate by spectrophotometric analysis to determine antibody concentration and dye-to-antibody ratio; and
(k) applying the formulated antibody conjugate to a biological sample and sequentially imaging the biological sample using a plurality of microscopy modalities selected from a group comprising Widefield Fluorescence Microscopy, Widefield Structured Illumination Microscopy (SIM), Confocal Fluorescence Microscopy, super resolution Airyscan Fluorescence Microscopy, Lattice SIM, Lattice SIM2, and Stochastic Optical Reconstruction Microscopy.
